(19) United States
(12) Patent Application Publication
(10) Pub. No.: US 2025/0276115 A1
(43) Pub. Date: Sep. 4, 2025

(54) SYSTEMS AND METHODS FOR MACHINE LEARNING-BASED DIALYSIS CARTRIDGE RECHARGE

(71) Applicant: Mozarc Medical US LLC, Minneapolis, MN (US)

(72) Inventors: Megha Shankaralingappa, Chitradurga (IN); Murugavel Swaminathan, Thanjavur (IN)

(21) Appl. No.: 19/065,706

(22) Filed: Feb. 27, 2025

(30) Foreign Application Priority Data
Feb. 29, 2024 (IN) 202411014981

(51) Int. Cl. A61M 1/16

(52) U.S. Cl. CPC A61M 1/1607; A61M 1/1696; A61M 2205/3334

(57) ABSTRACT

Systems, devices and/or methods include receiving, by at least one processor, current sorbent material module recharge characteristics associated with a sorbent material module recharge of a sorbent material module configured for dialysis therapy of a patient. The at least one processor may input the current sorbent material module recharge characteristics into a recharge machine learning model to output a recharge completion estimation of the sorbent material module based at least in part on recharge machine learning model parameters, wherein the recharge completion estimation represents a degree of sorbent material module recharge being complete. The at least one processor may control a sorbent material module recharge pump to vary the current sorbent material module recharge characteristics based at least in part on the recharge completion estimation.

### FIELD OF TECHNOLOGY

The present disclosure generally relates to computer-based systems and methods for machine learning (ML) based dialysis cartridge recharge, including leveraging ML cartridge state prediction.

### BACKGROUND OF TECHNOLOGY

One or more sorbent(s) are used for sorbent dialysis to remove waste and unwanted solutes including ammonium, potassium, calcium, and magnesium ions from dialysate. The sorbents are generally packaged in a sorbent module. Known recharging systems do not control the volume of chemical solutions used in recharging the sorbents, and instead simply treat the materials with enough recharging chemicals to ensure complete recharging. Complete recharging of the sorbent materials is generally used to cover worst case situations to avoid ammonia breakthrough in patients with high levels of urea or other ions. Complete recharging of the sorbent materials in each case is wasteful and more costly than recharging the sorbent materials only to the point necessary for a future dialysis session. Recharging sorbent material or zirconium oxide in this fashion results in the use of higher volumes of recharging chemicals than may be necessary.

### DEFINITIONS

Unless defined otherwise, all technical and scientific terms used have the same meaning as commonly understood by one of ordinary skill in the art.

The articles “a” and “an” are used to refer to one or to over one (i.e., to at least one) of the grammatical object of the article. For example, “an element” means one element or over one element.

The term “acid concentration” refers to the number of moles of an acid dissolved in a given volume of water.

The term “acid solution” refers to an aqueous solution having a pH less than 7.

An “acid source” is a fluid or concentrate source from which an acid solution can be obtained.

The term “ammonia breakthrough” refers to ammonia in a fluid exiting a sorbent module.

The term “amount of cations removed by the sorbent material module in a dialysis session” refers to the total number of moles of potassium, calcium, magnesium, ammonium, and other cations adsorbed by sorbent material in the sorbent material module during dialysis therapy.

The term “average dialysis session length” refers to the amount of time a patient spends undergoing dialysis in a normal dialysis session.

The term “average number of dialysis sessions per week” refer to the number of times a patient undergoes dialysis treatment during a normal treatment schedule.

The term “base concentration” refers to the number of moles of a base dissolved in a given volume of water.

The term “base solution” refers to an aqueous solution having a pH of greater than 7.

The term “base source” is a fluid or concentrate source from which a base solution can be obtained.

The term “blood flow rate” refers to an amount of blood pumped through an extracorporeal circuit in a given period of time.

The term “blood leak” refers to blood of the patient crossing a dialyzer membrane into a dialysate.

The term “brine source” refers to a source of a solution of salts and/or buffers containing solutes used in recharging a sorbent material. In certain embodiments, the brine source can contain a sodium salt, acetic acid, sodium acetate, or combinations thereof.

The term “buffer solution” refers to an aqueous solution containing a weak acid and the conjugate base of the weak acid.

The term “clearance” refers to a rate at which solutes pass through a dialyzer membrane.

The term “comprising” includes, but is not limited to, whatever follows the word “comprising.” Use of the term indicates the listed elements are required or mandatory but that other elements are optional and may be present.

The term “concentration” refers to an amount of a solute per a given volume of a solvent.

The term “consisting of” includes and is limited to whatever follows the phrase “consisting of.” The phrase indicates the limited elements are required or mandatory and that no other elements may be present.

The term “consisting essentially of” includes whatever follows the term “consisting essentially of” and additional elements, structures, acts or features that do not affect the basic operation of the apparatus, structure or method described

The terms “control,” “controlling,” or “controls” refers to the ability of one component to direct the actions of a second component.

A “control system” can be a combination of components that act together to maintain a system to a desired set of performance specifications. The control system can use processors, memory and computer components configured to interoperate to maintain the desired performance specifications. The control system can also include fluid or gas control components, and solute control components as known within the art to maintain the performance specifications.

The term “desired initial therapy sorbent material effluent pH” refers to an initial sorbent material effluent pH during therapy set or determined, at least in part, on the needs and capabilities of the system and patient.

The terms “determining” and “determine” refer to ascertaining a particular state or desired state of a system or variable(s).

The term “dialysate flow rate” refers to an amount of dialysate pumped through a dialysate flow path in a given period of time.

The term “dialysis prescription” refers to dialysis parameters intended to be used during a dialysis session. In certain embodiments, a “dialysis prescription” can refer to an intended concentration of one or more solutes in the dialysate used during treatment. For example, a calcium dialysis prescription can refer to the intended calcium concentration of the dialysate during treatment.

A “dialysis session” is time period that a patient is treated by dialysis, hemodialysis, hemofiltration, ultrafiltration, or other blood fluid removal therapy.

A “dialysis session parameter” is any data or condition relating to a specified dialysis session.

The term “dialysis time” refers to the length of time of a specific dialysis session.

The term “dialyzer size” refers to a surface area of a dialyzer membrane in a dialyzer.

The term “dialyzer type” refers to whether a specific dialyzer is a high-flux or low-flux dialyzer. The type can include other characteristics of properties of the dialyzer in addition to flux such as efficiency and membrane types. Efficiency properties such as membrane size (surface area), porosity, thickness, internal fiber diameters, and design (wavelike, straight fiber) are contemplated.

A “disinfectant source” can refer to a fluid source capable of destroying or removing biological contaminants.

The term “direct measurement” refers to using a sensor or other system to determine one or more parameters.

The term “duration to a next dialysis session” refers to an estimated length of time between the end of a first dialysis session for a patient and the beginning of a second dialysis session for the same patient.

“Estimated,” to “estimate,” or “estimation” refer to a determination of one or more parameters indirectly using one or more variables.

The term “fluidly connectable” refers to the ability of providing for the passage of fluid, gas, or combination thereof, from one point to another point. The ability of providing such passage can be any connection, fastening, or forming between two points to permit the flow of fluid, gas, or combinations thereof. The two points can be within or between any one or more of compartments of any type, modules, systems, components, and rechargers.

The term “fluidly connected” refers to a particular state such that the passage of fluid, gas, or combination thereof, is provided from one point to another point. The connection state can also include an unconnected state, such that the two points are disconnected from each other to discontinue flow. It will be further understood that the two “fluidly connectable” points, as defined above, can from a “fluidly connected” state. The two points can be within or between any one or more of compartments, modules, systems, components, and rechargers, all of any type.

The term “fluid removed during a session” refers to the total amount of fluid removed from the blood of a patient during a dialysis session.

A “heater” is a component capable of raising the temperature of a substance, container, or fluid.

The term “heating” or to “heat” refers to raising the temperature of a material.

The term “hypotensive episode” refers to an instance of low blood pressure in a patient during treatment.

The term “inlet” of a sorbent module can refer to a portion of a sorbent module through which fluid, gas, or a combination thereof can be drawn into the sorbent module.

The term “initial therapy sorbent material effluent pH” refers to the pH of a fluid exiting a sorbent material sorbent module at or near the beginning of therapy.

The term “mixing” or to “mix” generally refers to causing or more fluids from any source to combine together. For example, “mixing” can include laminar or turbulent flow at a location in a fluid line or a junction. Another example of “mixing” can include receiving one or more fluids in a component configured to receive fluids from one or multiple sources and to mix the fluids together in the component. Additionally, mixing can refer to the dissolution of a solid or solids with a fluid, wherein the solid or solids is dissolved in the fluid.

The term “necessary to recharge” a sorbent material refers to an amount of one or more recharge solutions required to result in a sorbent material with a specified functional capacity. In certain embodiments the specified functional capacity can be near 100% or can be lower depending on the needs of a patient.

The term “number of previous dialysis sessions” can refer to any number of dialysis sessions for a patient. The number of previous dialysis sessions can be 1, 2, 3, or more dialysis sessions.

A “patient” or “subject” is a member of any animal species, preferably a mammalian species, optionally a human. The subject can be an apparently healthy individual, an individual suffering from a disease, or an individual being treated for a disease. In certain embodiments, the patient can be a human, sheep, goat, dog, cat, mouse or any other animal.

The term “patient acidotic state” refers to a pH level in the blood of a patient.

A “patient parameter” is any data that gives relevant information about the health status and therapy requirements of a patient.

“Patient residual kidney function” is a measurement of how well a kidney of a patient is working as compared to a healthy individual.

“Patient volume” refers to the total amount of water in a patient.

“Patient weight” refers to the mass of a patient.

The term “pH alarm” refers to an indication that the pH of a fluid is outside of a predetermined range.

The term “pH of the sorbent material” refers to the negative log of the concentration of hydrogen ions absorbed onto a given amount of sorbent material.

“Phosphate bleed” refers to an amount of phosphate ions originally present as sorbent material that leak into a fluid pumped through a sorbent module.

“Pre-dialysis patient cation measurements” refer to determinations of cation levels in a patient prior to a dialysis session.

“Pre-dialysis patient total CO2 measurements” refer to determinations of total CO2 levels in a patient prior to a dialysis session, and can include carbon dioxide levels, bicarbonate levels, and carbonate levels.

“Pre-dialysis patient phosphate measurements” refer to a determination of a phosphate level in a patient prior to a dialysis session.

The term “pre-dialysis patient level,” when referring to specific solutes or materials, refers to the concentration of the solutes or materials in the blood of a patient prior to a dialysis session.

The term “pump” refers to any device that causes the movement of fluids or gases by applying suction or pressure.

The terms “pumping,” “pumped,” or to “pump” refers to moving a fluid, a gas, or a combination thereof with a pump.

A “receiving compartment” is a space within a recharger into which a sorbent module to be recharged is placed.

A “recharge solution” is a solution containing appropriate ions for recharging a specific sorbent material. A recharge solution can be a single solution containing all necessary ions for recharging a sorbent material. Alternatively, the recharge solution can contain some of the ions for recharging the sorbent material, and one or more other recharge solutions can be used to recharge the sorbent material.

A “recharge solution source” is any fluid or concentrate source from which a recharge solution can be obtained.

“Recharging” refers to treating a sorbent material to restore the functional capacity of the sorbent material so as to put the sorbent material back into a condition for reuse or use in a new dialysis session. In some instances, the total mass, weight and/or amount of “rechargeable” sorbent materials remain the same. In some instances, the total mass, weight and/or amount of “rechargeable” sorbent materials change. Without being limited to any one theory of invention, the recharging process may involve exchanging ions bound to the sorbent material with different ions, which in some instances may increase or decrease the total mass of the system. However, the total amount of the sorbent material will in some instances be unchanged by the recharging process. Upon a sorbent material undergoing “recharging,” the sorbent material can then be said to be “recharged.”

A “recharging flow path” is a path through which fluid can travel while recharging sorbent material in a reusable sorbent module.

The term “salt concentration,” as used herein, refers to the number of moles of a sodium salt dissolved in a given volume of water.

A “salt solution” refers to an aqueous solution containing dissolved sodium and counter ions.

A “salt source” is a fluid or concentrate source from which a salt solution can be obtained.

The term “sequential order” refers to two or more events occurring at different times, as opposed to simultaneously.

The term “session time” refers to a length of time of a dialysis session, from the beginning of dialysis treatment of a patient to the end of the dialysis treatment.

The terms “set based at least in part on” or “set based on” refer to a calculation of a parameter value, wherein the value is a function of at least one other variable.

A “sorbent module” or “sorbent module” means a discreet component of a sorbent module. Multiple sorbent modules can be fitted together to form a sorbent module of two, three, or more sorbent modules. In some embodiments, a single sorbent module can contain all of the necessary materials for dialysis. In such cases, the sorbent module can be a “sorbent module.” The “sorbent module” or “sorbent module” can contain any material for use in sorbent dialysis and may or may not contain a “sorbent material” or adsorbent. In other words, the “sorbent module” or “sorbent module” generally refers to the use of the “sorbent module” or “sorbent module” in sorbent-based dialysis, e.g., REDY (REcirculating DYalysis), and not that a “sorbent material” is necessarily contained in the “sorbent module” or “sorbent module.”

The term “sorbent material” refers to a material capable of removing specific solutes from a fluid. In certain embodiments, the sorbent material can be zirconium oxide or other sorbent material.

The phrase “specific to a recharge process being used” can refer to one or more variables that are used to recharge a sorbent material. In certain embodiments, the variables can include a composition of a recharge solution, concentrations of one or more solutes in the recharge solution, temperature of the recharge solution, or flow rate of the recharge solution.

The term “specified temperature” is a temperature range calculated or determined prior to recharging a sorbent material module.

The term “starting water quality” can refer to the quality of the water used in preparing an initial dialysate for a dialysis session. In certain embodiments, the starting water quality can refer to an amount of solutes dissolved in the water used in preparing the initial dialysate.

A “static mixer” is a component configured to receive fluids from one or multiple sources and to mix the fluids together. The static mixer may include components that agitate the fluids to further mixing.

The term “temperature sensor” refers to a device for measuring the temperature of a gas or liquid in a vessel, container, or fluid line.

The term “time averaged volume flow rate” can refer to a volume of fluid moved per unit time averaged over a dialysis session.

The term “total cation” can refer to an amount of cations in a dialysate throughout a dialysis session.

“Total CO2” can refer to the total amount of carbon dioxide, bicarbonate ions, and carbonate ions in a dialysate throughout a dialysis session.

The term “total phosphate” refers to the total amount of phosphate ions in a dialysate throughout a dialysis session.

The term “total volume treated” refers to a total amount of fluid pumped through a sorbent module or sorbent module during dialysis treatment.

The term “ultrafiltration rate” refers to an amount of fluid removed from the blood of a patient in a given period of time.

“Urea reduction ratio” or “URR” refers to the amount by which the urea level of a patient is reduced during treatment. The URR can be expressed as I minus the ratio of the patient's ending urea level over the patient's starting urea level.

“URR achieved” refers to the urea reduction level actually resulting from a dialysis session. “URR target” refers to an intended urea reduction ratio during a dialysis session.

A “valve” is a device capable of directing the flow of fluid or gas by opening, closing or obstructing one or more pathways to control whether or not the fluid or gas to travel in a particular path. One or more valves that accomplish a desired flow can be configured into a “valve assembly.”

The term “volume” refers to a three-dimensional amount of space occupied by a material.

A “water source” is a fluid source from which water can be obtained.

“Zirconium oxide” is a sorbent material that removes anions from a fluid, exchanging the removed anions for different anions. Zirconium oxide may also be referred to as hydrous zirconium oxide,

“Zirconium phosphate” is a sorbent material that removes cations from a fluid, exchanging the removed cations for different cations

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments

FIG. 1 depicts a recharging flow path for recharging a sorbent module with two recharge solution sources in accordance with one or more embodiments of the present invention.

FIG. 2 depicts a recharging flow path for recharging a sorbent module with a single recharge solution source in accordance with one or more embodiments of the present invention.

FIG. 3 depicts a recharging flow path for recharging a zirconium oxide sorbent module with a single recharge solution source in accordance with one or more embodiments of the present invention.

FIG. 4 depicts a recharge control system for recharging a sorbent module according to ML-based prediction of the sorbent module state in accordance with one or more embodiments of the present disclosure.

FIG. 5 is a flow diagram illustrating a recharge control method for recharging a sorbent module according to ML-based prediction of the sorbent module state in accordance with one or more embodiments of the present disclosure.

FIG. 6 depicts a block diagram of an exemplary computer-based system and platform for recharging a sorbent module according to ML-based prediction of the sorbent module state in accordance with one or more embodiments of the present disclosure.

### DETAILED DESCRIPTION

Various detailed embodiments of the present disclosure, taken in conjunction with the accompanying FIGs., are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an.” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

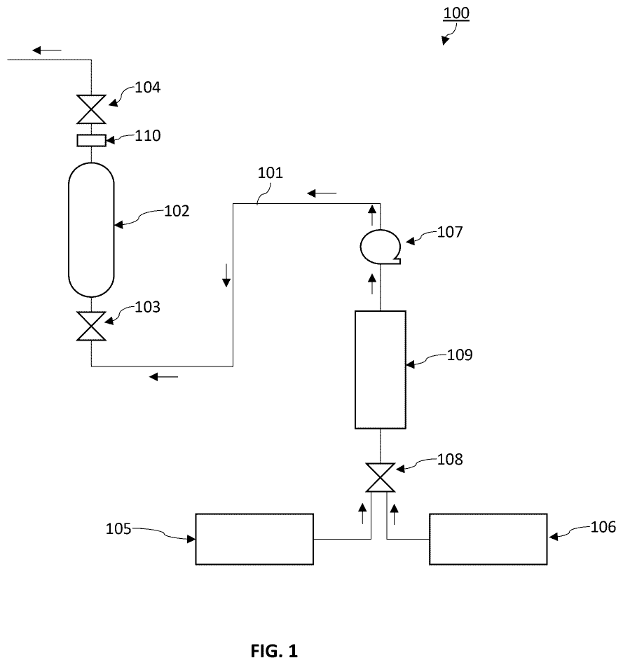

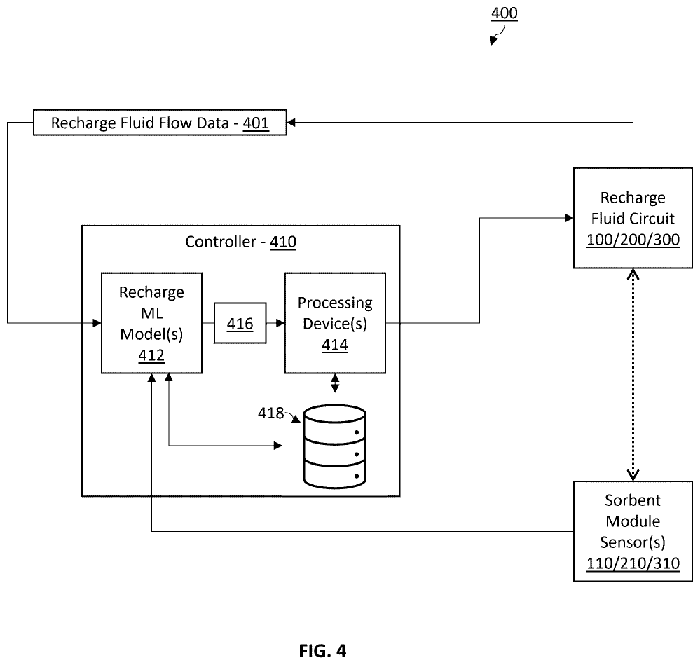

FIGS. 1 through 6 illustrate systems and methods of improved sorbent module recharge by leveraging ML-based measurement of sorbent module state in real-time during sorbent module recharge. The following embodiments provide technical solutions and technical improvements that overcome technical problems, drawbacks and/or deficiencies in the technical fields involving recharging of sorbent modules for dialysis systems, where typical sorbent module recharging including using fluid flow rate over a period of time of recharge using a recharge solution to accomplish recharging with a rough estimate based on the fluid flow rate. Such techniques do not take into account the state of the sorbent module being recharged and thus only roughly estimate how long to supply the recharge solution, with a bias towards oversupply, thus increasing time and costs without achieving any additional benefit. As explained in more detail, below, technical solutions and technical improvements herein include aspects of improved recharge solution supply by using sensed material quantity in the sorbent module and one or more ML models to estimate a state of the sorbent module, thus enabling real-time determination of the degree of recharge of the sorbent module for faster and more accurate sorbent module recharge, thus preserving material and reducing time to completion of the sorbent module recharge. Based on such technical features, further technical benefits become available to users and operators of these systems and methods. Moreover, various practical applications of the disclosed technology are also described, which provide further practical benefits to users and operators that are also new and useful improvements in the art.

In some embodiments, a recharger for recharging sorbent material in a sorbent module may include at least a first receiving compartment for receiving a sorbent module. The receiving compartment has a sorbent module inlet and a sorbent module outlet fluidly connectable to an inlet and outlet of a sorbent module. A door may control access to the receiving compartment. A user interface can receive information from a user for controlling the recharge process. The recharger can optionally include a second receiving compartment for receiving a second sorbent module, containing the same or a different sorbent material for concurrent recharging of sorbent materials. The recharger can include any number of receiving compartments for receiving multiple sorbent modules or various combinations of sorbent modules. The recharger can have 1, 2, 3, 4, 5, or more receiving compartments for recharging any number of sorbent modules. The recharger can be fluidly connectable to one or more recharge solution sources through a recharging flow path. Pumps and valves may control the movement of fluid from the recharge solution sources through the sorbent material module.

Sorbent material is recharged by pumping one or more solutions including acids, bases, and sodium salts through the sorbent material module. The hydrogen and sodium ions in the recharge solutions displace ions such as, e.g., potassium, calcium, magnesium, ammonium, and other ions from either the dialysate or source water that are bound and adsorbed by the sorbent material during use. The recharged sorbent material with sodium and hydrogen ions can be used during dialysis to remove cation solutes from the used dialysate. For example, zirconium oxide can be recharged by pumping one or more solutions containing a hydroxide base through the zirconium oxide sorbent module. The hydroxide ions can displace phosphate ions that are bound and adsorbed by the zirconium oxide during use.

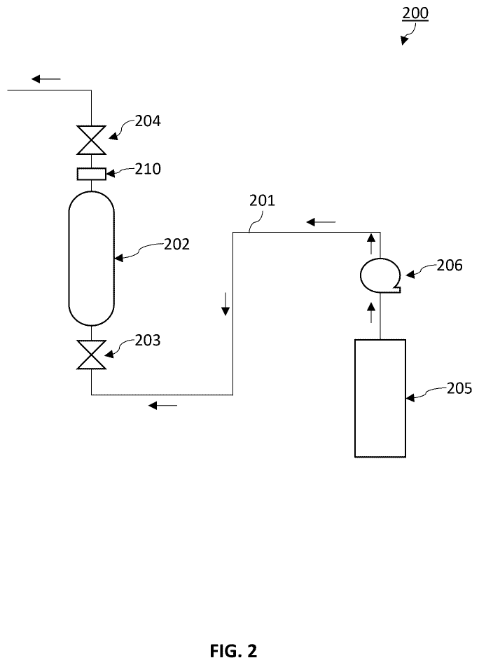

The initial therapy sorbent material effluent pH depends on the ratio of hydrogen to sodium ions on the sorbent material. FIG. 2 illustrates the effect of the recharge solution pH on the initial therapy sorbent material effluent pH. The recharge solutions may each contain mixtures of sodium chloride, sodium acetate, and acetic acid. In some embodiments, the pH of the recharge solution controls the initial therapy sorbent material effluent pH. In some embodiments, the initial therapy sorbent material effluent pH can be controlled by adjusting the pH of the recharge solution to result in a desired initial therapy sorbent material effluent pH. The initial therapy sorbent material effluent pH is controlled by altering a ratio of hydrogen ions to sodium ions in the sorbent material. A lower pH recharge solution increases the hydrogen ion to sodium ion ratio on the recharged sorbent material and lowers the initial therapy sorbent material pH. A higher pH recharge solution decreases the hydrogen ion to sodium ion ratio on the recharged sorbent material and increases the initial therapy sorbent material effluent pH. The sorbent material effluent pH can be customized based on the needs of the user by controlling the pH of the recharge solution.

In some embodiments, the initial sorbent material effluent pH is determined by the recharge solutions used. The pH profile of the sorbent material depends on the mass of the sorbent material and the mass of bicarbonate pumped through the sorbent material module. With a high sorbent material mass, and a low bicarbonate mass pumped through the sorbent material, the initial sorbent material effluent pH may be maintained for an entire therapy session. The sorbent material acts like a buffer and as more bicarbonate is pumped through the sorbent material, the buffer capacity becomes exceeded and the pH will start to increase.

Table 1 provides non-limiting examples of recharge solutions and the resulting initial therapy sorbent material effluent pH. In each case, the recharge solution was heated to 80° C. prior to use. A higher ratio of sodium acetate to acetic acid results in a higher recharge solution pH, and therefore a higher initial therapy sorbent material effluent pH. The relative amounts of acid, base, and sodium salt can be set to generate a recharge solution having the desired pH.

TABLE 1

| | | | | | | Initial |
|---|---|---|---|---|---|---|
| | Total Na | | NaAce | | | Effluent |
| Solution | (M) | NaCl (M) | (M) | HAce (M) | pH | pH |
| 1 | 4.00 | 4.00 | 0.00 | 0.20 | 2.75 | 4.40 |
| 2 | 4.00 | 3.98 | 0.02 | 0.20 | 3.74 | 5.42 |
| 3 | 4.00 | 3.88 | 0.12 | 0.40 | 4.23 | 5.99 |
| 4 | 4.00 | 3.60 | 0.40 | 0.40 | 4.75 | 6.58 |

Each of the recharge solutions may be combinations of sodium chloride, sodium acetate, and acetic acid. One of skill in the art will understand other buffer combinations can be used in place of sodium acetate and acetic acid, including sodium citrate and citric acid, glycolic acid and sodium glycolate, propionic acid and sodium propionate, phosphoric acid and sodium phosphate, or any combination thereof. The relative amounts of sodium chloride and buffer to achieve a desired initial therapy sorbent material effluent pH will depend on the pKa of the acid used and can be varied as needed.

In some embodiments, the sorbent material module effluent pH affects the amount of bicarbonate needed during dialysis. Urease in the sorbent module converts urea to carbon dioxide and ammonium ions. The carbon dioxide produced is in equilibrium with bicarbonate in the dialysate. The carbon dioxide must be removed from the dialysate by a degasser prior to the dialysate entering the dialyzer. The degasser can be any type of degasser known in the art for use in dialysis systems. A high sorbent material effluent pH during therapy drives the equilibrium towards bicarbonate formation, resulting in too much bicarbonate in the dialysate for safe treatment. A low sorbent material effluent pH during therapy drives the equilibrium towards carbon dioxide formation, requiring addition of bicarbonate to the dialysate and placing a high burden on the degasser. One type of degasser suitable for removing carbon dioxide is a membrane contactor. A membrane contractor is a dual chamber device with a hydrophobic microporous membrane separating the chambers. The hydrophobic microporous membrane allows gas transport without allowing water transport across the membrane. Liquid containing gas—such as CO₂—is passed on one side of the membrane and either inert gas or a vacuum is applied to the chamber on the opposite side of the membrane. CO₂ is transported from the liquid by diffusion. Another example of a degasser is a vacuum degasser. A vacuum degasser is a chamber in which a vacuum can be applied, and which is fluidly connected to a liquid containing gas to be removed. The liquid is sprayed or atomized in the vacuum chamber. The high surface area of the liquid droplets allows efficient removal of the gas. The sorbent material effluent pH can be controlled by the pH of the recharge solution to meet the needs of the patient and system. The sorbent material effluent pH is a function of the pH, pKa, buffer capacity, sodium chloride level, and temperature of the recharge solution. As described, a control system can automatically determine the volumes of each component needed to achieve a desired initial therapy sorbent material effluent pH based on each of the factors and/or one or more machine learning models for estimating the state of the state of the cartridge.

In some embodiments, a sorbent material effluent pH of about 6.5 allows greater than 95% of patients to be treated with a dialysate bicarbonate concentration of 25 mM. At a higher pH, fewer patients can be treated. For example, only about 40% of patients can be treated with a sorbent material effluent pH of 6.9 and a dialysate bicarbonate level of 25 mM. At a very low pH, too much acid is added to the dialysate by the sorbent material, and additional bicarbonate will be necessary to keep the dialysate pH within a safe range, and a degasser is needed to remove carbon dioxide. The initial therapy sorbent material effluent pH can be set at any value capable of generating safe dialysate, including between 4.0 and 6.9. A dialysate with a lower pH places a higher burden on the degasser.

In some embodiments, any combination of acid, base, and sodium salt capable of generating a recharge solution within the desired pH range can be used in recharging the sorbent material. Non-limiting examples of acids and bases include sodium acetate and acetic acid, sodium citrate and citric acid, glycolic acid and sodium glycolate, propionic acid and sodium propionate, phosphoric acid and sodium phosphate, or any combination thereof. In some embodiments, the relative amounts of acid and base needed to generate a recharge solution with a desired pH will vary with the pKa of the acid. The relative volumes of the acid and base can be varied based on the pKa of the particular acid and base used. For example, a recharge solution with 3.1 M sodium chloride, 0.9 M sodium acetate, and 0.6 M acetic acid has a pH of 4.6, which will generate a sorbent material effluent pH of 6.5.

As described, the sorbent material effluent pH during therapy controls the equilibrium between carbon dioxide and bicarbonate in the dialysate. Carbon dioxide and bicarbonate in the dialysate generally may come from two sources, the conversion of urea to carbon dioxide and any bicarbonate added to the dialysate. To minimize the amount of additional bicarbonate required, the sorbent material effluent pH can be set to a higher value, at least for patients that can be effectively treated with a higher dialysate bicarbonate level. The higher sorbent material effluent pH during therapy drives the bicarbonate/carbon dioxide equilibrium towards bicarbonate formation, retaining bicarbonate generated from the urea removed from the patient.

In some embodiments, a control system in the recharger can determine the optimal initial therapy sorbent material effluent pH for a patient based on the patient's pre-treatment bicarbonate and urea levels. For alkalotic patients, a lower initial therapy sorbent material effluent pH can be selected to minimize the amount of bicarbonate formed from the patient's urea. For other patients, a higher initial therapy sorbent material effluent pH can be selected to generate a higher amount of bicarbonate from the patient's urea, reducing the additional bicarbonate needed and minimizing the burden on the degasser. Alternatively, a user interface can be provided, with the user directly inputting the desired initial therapy sorbent material effluent pH.

The control system can be any component capable of monitoring and affecting the states of the recharger. The control system can use processors, memory and computer components to carry out the functions described. The control system is in communication with the pumps and valves of the recharging flow paths and can control the pumps and valves in accordance with stored instructions. The control system is also in communication with various sensors in the recharging flow paths. The control system receives data from the sensors and controls the pumps and valves of the recharging flow path on the basis of the data in accordance with stored instructions. Factors affecting the desired initial therapy sorbent material effluent pH, such as patient pre-treatment urea and bicarbonate levels can be communicated to the control system by any means known in the art. The control system can automatically determine the optimal recharging solution pH using mathematical algorithms or look-up tables and operate the pumps and valves of the recharging flow paths to control the recharging process.

FIG. 1 illustrates a non-limiting embodiment of a recharging flow path 101 for customization of a recharging solution. A sorbent material module 102 can connect to the recharging flow path 101 through sorbent material inlet 103 and sorbent material outlet 104. The sorbent material inlet 103 and/or the sorbent material outlet 104 may include one or more valves to control flow into and/or out of the sorbent material module 102. A sorbent module sensor 110 may be positioned at the sorbent material outlet 104. The sorbent module sensor 110 can include one or more sensor devices configured to measure characteristics of the sorbent material effluent exiting the sorbent material module 102 during recharging. For example, the sorbent module sensor 110 may detect the sorbent material effluent pH, the presence and/or concentration of one or more materials and/or minerals (e.g., salts, phosphate, urea, etc.), presence and/or quantity of one or more microbes, among other characteristics of the effluent.

Pump 107 provides a driving force for moving fluids through the recharging flow path 101. A salt or brine source 105, containing a salt solution such as sodium chloride or mixtures of sodium chloride and sodium acetate, and an acid source 106 containing an acid solution, such as acetic acid, are fluidly connected to the recharging flow path 101. Valve 108 determines the amount of each recharge solution that enters the recharging flow path 101 to generate a recharge solution having a specified acid concentration, base concentration, and salt concentration, and can be controlled by the control system. The salt solution from brine source 105 is pumped through the recharging flow path 101 to the sorbent material module 102. Acid from acid source 106 can be pumped into the recharging flow path 101 at a ratio to the salt solution based on the desired recharge solution pH. For example, acetic acid from acid source 106 can be metered into the salt solution in recharging flow path 101 at a specified rate to control the pH of the resulting recharge solution. A higher salt solution to acid ratio will result in a recharge solution at a higher pH, while a lower salt solution to acid ratio will result in a recharge solution at a lower pH. The control system can automatically control valve 108 to control the ratio of sodium chloride to acid. Alternatively, the acid source 106 can contain a buffer solution, such as sodium acetate and acetic acid, and the control system can control the ratio of salt solution and buffer to control the recharge solution pH. A static mixer 109 can be included to ensure complete mixing of the acid and salt solutions. Alternatively, the acid and salt solutions can be mixed through the mixing of the two fluid streams in the recharging flow path 101. One of skill in the art will understand that different pump and valve arrangements can be used with the system illustrated in FIG. 1. For example, the brine source 105 and acid source 106 can be connected to the recharging flow path 101 through separate pumps, allowing simultaneous addition of salt solution and acid to the recharging flow path 101.

Alternatively, a system as illustrated in FIG. 1 can have a salt solution and an acid in a first recharge solution source, with a base solution, such as sodium hydroxide, in a base source. The salt solution and acid can be pumped through the sorbent material module, with the base solution metered in to generate a recharge solution with the desired pH in situ.

For example, the recharging flow path 101 in FIG. 1 can also recharge the sorbent material module 102 by addition of recharging solutions in a sequential order. The acid solution from acid source 106 can be pumped through the sorbent material module 102 first, followed by sodium chloride and sodium acetate from brine source 105. The initial acid solution will generate a sorbent material module 102 at a low pH, and the later addition of sodium chloride and sodium acetate will raise the pH as sodium ions displace the hydrogen ions initially adsorbed by the sorbent material. The resulting sorbent material effluent pH will depend on the amount of sodium chloride and sodium acetate pumped through the sorbent material module 102 in the second step. The control system can control the sodium chloride and sodium acetate addition to generate a sorbent material module 102 with the desired initial therapy sorbent material effluent pH. The sensor 110 including a pH sensor can be placed in the sorbent material effluent to determine the sorbent material effluent pH, and the sodium chloride can be stopped when the pH sensor reads the desired pH. The concentration and amount of sodium chloride and sodium acetate pumped through the sorbent material module 102 will control the initial therapy sorbent material effluent pH after recharging. Alternatively, the sodium chloride and sodium acetate can be pumped through the sorbent material module 102 first, followed by the acid.

FIG. 2 illustrates a recharging flow path 201 with a single recharge solution source 205 containing a sodium salt and buffer. A sorbent material module 202 can be fluidly connected to the recharging flow path 201 through sorbent material inlet 203 and sorbent material outlet 204. A sorbent module sensor 210 may be positioned at the sorbent material outlet 204. The sorbent module sensor 210 can include one or more sensor devices configured to measure characteristics of the sorbent material effluent exiting the sorbent material module 202 during recharging. For example, the sorbent module sensor 210 may detect the sorbent material effluent pH, the presence and/or concentration of one or more materials and/or minerals (e.g., salts, phosphate, urea, etc.), among other characteristics of the effluent.

Pump 206 provides a driving force for moving fluids through the recharging flow path 201. Recharge solution source 205 is fluidly connected to the recharging flow path 201. A recharge solution in recharge solution source 205 at the desired recharge solution pH can be pumped through the sorbent material module 202 to recharge the sorbent material. To alter the initial therapy sorbent material effluent pH, the pH of the recharge solution can be altered. The user can add solid or concentrated sources of an acid, a base, a salt, or combinations thereof, to control the pH of the recharge solution to generate a recharge solution having a specified acid concentration, base concentration, and salt concentration. The control system can inform the user of the correct amounts of acid, base, or salt to add to the recharge solution source 205. Alternatively, a separate source of acid, base, or salt can be included in the recharger, and the system can automatically add the correct amount to the recharge solution source 205 based to generate a recharge solution with the desired pH. For example, a recharge solution with a pH of 4.6 can be placed in the recharge solution source 205 and used for the majority of patients. For severely alkalotic patients, the system or user can add a predetermined amount of acid to lower the recharge solution pH. To reduce the amount of bicarbonate needed during therapy, the system or user can add a predetermined amount of base to raise the recharge solution pH.

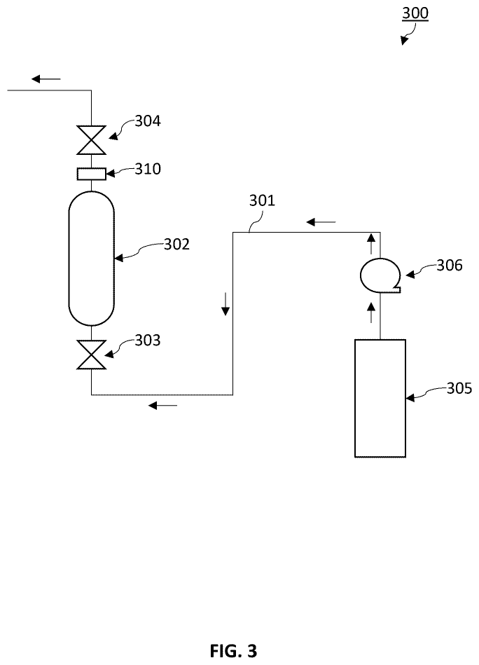

FIG. 3 illustrates a non-limiting embodiment of a recharging flow path 301 for recharging zirconium oxide in a reusable zirconium oxide sorbent module 302. The sorbent module 302 can be fluidly connected to the recharging flow path 301 through sorbent module inlet 303 and sorbent material module outlet 304. A sorbent module sensor 310 may be positioned at the sorbent material outlet 304. The sorbent module sensor 310 can include one or more sensor devices configured to measure characteristics of the sorbent material effluent exiting the sorbent material module 302 during recharging. For example, the sorbent module sensor 310 may detect the sorbent material effluent pH, the presence and/or concentration of one or more materials and/or minerals (e.g., salts, phosphate, urea, etc.), among other characteristics of the effluent.

Pump 306 provides a driving force for moving fluids through the recharging flow path 301. Recharge solution source 305 is fluidly connected to the recharging flow path 301 and can be a base source. The recharge solution source 305 can include a solution such as sodium hydroxide at a specified concentration. A control system (not shown) can set the volume of sodium hydroxide pumped through the sorbent module 302 based on one or more patient parameters and/or one or more dialysis session parameters.

In some embodiments, the recharging flow paths illustrated in FIGS. 1-3 can include additional fluid sources. A water source can provide water for flushing and rinsing of the sorbent material module before and after recharging. A water source can also provide in-line dilution of any of the recharge solutions, allowing a more concentrated recharge solution in the recharge solution sources. A disinfectant source can provide a disinfection solution for disinfecting the sorbent material module prior to recharging. The disinfection solution can be any solution capable of disinfecting the sorbent material sorbent module, including a peracetic acid solution, a citric acid solution, or any other disinfectant.

The recharger can include multiple recharging flow paths for recharging multiple sorbent modules. For instance, a single recharger can include two or more recharging flow paths for recharging two or more sorbent material sorbent modules. Additionally, a single recharger can include both of two sorbent material recharging flow paths for recharging both sorbent material sorbent modules. One or more recharge solution sources can be shared by both recharging flow paths, or separate recharge solution sources can be used in each flow path.

The total volume of recharge solution needed to recharge the sorbent material depends on the amount of cations removed by the sorbent material in the previous dialysis session and the amount of total CO₂ fed through the sorbent material during the previous dialysis session, which in turn depend on a number of patient and/or dialysis parameters. Patient parameters affecting the amount of cations removed by the sorbent material and the amount of CO₂ fed through the sorbent material include pre-dialysis patient potassium, calcium, magnesium, bicarbonate and urea levels; patient weight, patient volume, patient residual kidney function, an average number of dialysis sessions per week, patient acidotic state, and an average dialysis session length. Dialysis parameters affecting the amount of cations removed from the sorbent material include dialysate flow rate, blood flow rate, dialyzer size, dialyzer type, dialysis time, ultrafiltration rate, a potassium, calcium, magnesium, and bicarbonate dialysis prescription, whether ammonia breakthrough occurred, whether a pH alarm occurred, fluid removed during a session, total volume treated, starting water quality, URR target, URR achieved, clearance, whether a blood leak occurred, and whether a hypotensive episode occurred.

The total volume of recharge solution needed to recharge a sorbent such as zirconium oxide depends on the amount of phosphate removed by the sorbent in the previous dialysis session, which in turn can depend on a number of patient and/or dialysis parameters. Patient parameters affecting the amount of phosphate removed by the sorbent include patient weight, patient volume, patient residual kidney function, an average number of dialysis sessions per week, and an average dialysis session length. Dialysis parameters affecting the amount of phosphate removed by the sorbent include dialysate flow rate, blood flow rate, dialyzer size, dialyzer type, dialysis time, ultrafiltration rate, a potassium, calcium, magnesium, and bicarbonate dialysis prescription, whether ammonia breakthrough occurred, whether a pH alarm occurred, fluid removed during session, total volume treated, starting water quality, URR target, URR achieved, clearance, whether a blood leak occurred, and whether a hypotensive episode occurred.

Usage of a sorbent material module and/or a zirconium oxide module by a patient can be tracked with an RFID tag, barcode, or other tracking device. The recharge control system 400 can receive any one or more of the patient parameters influencing the amount of recharge solution needed and determine the necessary volume of the recharge solution for recharging the sorbent material module. In certain embodiments, the recharge control system 400 can receive patient and dialysis parameters from the prior usage and history of the sorbent module and patient to determine the necessary volume of the recharge solution for recharging the sorbent material module and/or zirconium oxide module. The recharge control system 400 can use all prior history and usage of the patient and sorbent modules, or can use the prior history and usage over any specified length of time. In certain embodiments, the recharge control system 400 can use the patient and dialysis parameters from a previous week, month, year, or longer period of time.

A tracking component, such as an RFID tag or bar code, can be affixed to the sorbent modules, and automatically read by the recharge control system 400 at various times, including prior to dialysis, after dialysis, prior to recharging, and after recharging. A single reader can read and track the sorbent modules at each stage of use, or separate readers can be included with the rechargers and dialysis systems to track usage of the sorbent modules. Other techniques and/or technologies for tracking the sorbent modules may be employed in the alternative or in any combination, such as, e.g., barcodes, quick response (QR) codes, serial numbers, near field communication (NFC) tags, Bluetooth™, or any other computer readable indicia or nay combination thereof.

The tracking system can track which patients used the sorbent modules and the dialysis parameters that affect the amount of recharge solutions necessary to recharge the sorbent modules. The parameters can be communicated to the recharge control system 400, which can then determine the amount of recharge solution necessary through ML-based estimation.

Generally, about 5.5 millimoles of sodium in the recharge solution is required per total milliequivalents of cations (ammonium+potassium+calcium+magnesium) that are fed through the sorbent material during therapy in order to recover greater than 90% of the original capacity of the sorbent material. Lower moles of sodium are needed per mole of cations loaded on the sorbent material for full recharging at elevated temperatures, and less recharge solution is needed with a higher recharge solution concentration. A higher amount of sodium may be needed if the recharging is conducted at room temperature. The recharge solution can have any amount of sodium ions relative to the amount of cations loaded on the sorbent material, including sodium ions between 5 and 15 times greater than the amount of cations loaded on the sorbent material. In certain embodiments, the total millimoles of sodium pumped through the sorbent material module during recharging can be between 5.0 and 15.0 millimoles of sodium per milliequivalent of cations, between 5.0 and 10.0 millimoles of sodium per milliequivalent of cations, between 5.0 and 6.0millimoles of sodium per milliequivalent of cations, between 7.0 and 12.0 millimoles of sodium per milliequivalent of cations, between 10.0 and 12.5 millimoles of sodium per milliequivalent of cations, or between 10.0 and 15.0 millimoles of sodium per milliequivalent of cations.

Generally, about 0.6-millimoles of total acetate (sodium acetate and acetic acid) in the recharge solution is required per millimole of total CO₂ (CO₂+HCO₃—+CO₃₂—) that is fed through the sorbent material during therapy in order to achieve the desired effluent pH profile during the next dialysis session. In certain embodiments, the total millimoles of acetate pumped through the sorbent material module during recharging can be between 0.3 and 1.0 millimoles of acetate per millimole of total CO₂, between 0.3 and 0.5 millimoles of acetate per millimole of total CO₂, between 0.5 and 0.7 millimoles of acetate per millimole of total CO₂, or between 0.6 and 1.0 millimoles of acetate per millimole of total CO₂.

As described, the patient acidotic state can be used to determine a desired sorbent material effluent pH for a future therapy session. For alkalotic patients, a lower initial therapy sorbent material effluent pH can be selected to minimize the amount of bicarbonate formed from the patient's urea. The initial therapy sorbent material effluent pH depends on the concentrations and volume of recharge solutions used, and can be used by the recharge control system 400 in determining a volume and/or concentration of the recharge solutions.

The amount of recharge solution needed can also depend on the temperature of the recharge solution. The recharging flow paths described can include a heater and optionally a heat exchanger for heating the recharge solution to a specified temperature prior to pumping the recharge solution through the sorbent material module, as recharging sorbent material may be more efficient at elevated temperatures. A temperature sensor determines the temperature of the recharge solution, and the recharge control system 400 can take temperature into account in determining the total amount of recharge solution necessary. The recharge solution can be heated to any specified temperature, including between 60-90° C., 60-70° C., 60-80° C., 75-85° C., or 80-90° C. During recharging, the recharge control system 400 can use only the volume of recharge solution necessary based on the total amount of cations loaded onto the sorbent material, the concentration of the recharge solution, and the temperature of the recharge solution, saving on costs and materials.

Generally, about 6.7 moles of sodium hydroxide is required to recharge, e.g., zirconium oxide per mole of phosphate fed through a zirconium oxide sorbent module during a previous dialysis session. In certain embodiments, the total moles of sodium hydroxide pumped through the zirconium oxide module during recharging can be between 5.0 and 8.4 moles of sodium hydroxide per mole of phosphate, between 5.0 and 6.5 moles of sodium hydroxide per mole of phosphate, between 6.0 and 7.0 moles of sodium hydroxide per mole of phosphate, between 6.0 and 8.0 moles of sodium hydroxide per mole of phosphate, or between 6.5 and 8.4 moles of sodium hydroxide per mole of phosphate.

The total cations and total CO₂ pumped through the sorbent material during a dialysis session can be obtained by direct measurement with one or more sensors in a dialysate flow path during treatment, or estimated. The estimates of total cations and total CO₂ can be based on patient weight, the dialysate prescription, pre-dialysis patient cation measurements and/or pre-dialysis patient total CO₂ measurements, or any combination thereof. Similarly, the total phosphate pumped through the zirconium oxide during a dialysis session can be obtained by direct measurement by a sensor in the dialysate flow path or estimated based on phosphate bleed from the sorbent material and pre-dialysis patient phosphate measurements. In certain embodiments, the total cations, total CO₂, and total phosphate pumped through the sorbent materials can be estimated based on a number of previous dialysis sessions. The values or parameters used can be tracked for a patient over any number of previous dialysis sessions and used to estimate the total cations and total CO₂ pumped through the sorbent material in the immediately prior dialysis session. The number of previous dialysis sessions used can be any number n of previous dialysis sessions, where n is 1 or greater. In certain embodiments, the number of previous dialysis sessions can be between 1-5, 2-10, 5-10, or greater number of dialysis sessions. The number of previous dialysis sessions can also be set as a length of time, including dialysis sessions over the prior week, month, year, or any other length of time.

A transport model across the dialyzer can include the dialysate flow rate, blood flow rate, dialyzer size and koA, pre-dialysis patient levels of the solutes, concentrations in the dialysate for the solutes, patient weight and volume, and ultrafiltration rate. The mass balance on the dialysate circuit can include the concentration of the solutes in the spent dialysate (determined from the dialyzer transport model), the volume of source water used, and the starting water quality, which includes the concentrations of the solutes in the starting water used to generate the initial dialysate.

The pre-dialysis patient levels of the solutes or the patient acidotic state can be measured with a blood analyzer before the treatment or can be estimated based on the patient's dialysis schedule and typical session parameters like frequency per week, session time, patient weight, patient acidotic state, and time since last session. The methods to estimate the concentration from these values can be derived by one skilled in the art.

As described, specific alarms or instances during treatment can also affect the concentration and volume of the recharge solution necessary for recharging the sorbent modules. Ammonia breakthrough could indicate that the sorbent material was fully loaded with cations, and the recharge volume may be increased accordingly. For example, if ammonia breakthrough occurs, 7.0 L of a recharge solution having 4.7 M NaCl, 0.4 M sodium acetate, and 0.4 M acetic acid can be used to recharge the sorbent material. Alternatively, the total concentrations of sodium and acetate in the recharge solution can be increased and the volume of recharge solution kept the same. In certain embodiments, the volume and/or concentration of the recharge solution can be increased beyond that required for recharging the sorbent material to ensure complete recharging.

A pH alarm could indicate that the sorbent material was fully titrated with bicarbonate during treatment, and the recharge solution volume or concentration should be increased accordingly. For example, 7.0 L of a recharge solution having 4.7 M NaCl, 0.4 M sodium acetate, and 0.4 M acetic acid can be used to recharge the sorbent material in the event of a pH alarm, or the concentrations of acetate in the recharge solution increased.

A blood leak could result in a need to increase the volume of disinfect solution needed to fully disinfect the sorbent material or zirconium oxide or to compensate for less efficient recharge due to more protein exposure of the sorbent material. In certain embodiments, 0.1 L extra of disinfectant solution can be used for each instance of a blood leak during treatment, depending on the disinfectant used and the concentration.

Accordingly, as detailed above, the state of the sorbent module during recharge is a highly multi-variate and dynamic analysis that would be difficult and computationally expensive (e.g., requiring processor and memory resources) to perform analytically. Therefore, the recharge control system 400 may employ one or more recharge ML models 412 to estimate a sorbent module state 416 from recharge fluid flow data 401 collected from the recharge fluid circuit 100, 200 and/or 300 detailed above. A processing device(s) 414 may control the recharge fluid circuit 100/200/300, e.g., by actuating one or more valves and/or pumps, to control the flow of recharge solution to the sorbent module based on the sorbent module state 416.

In some embodiments, the controller 410 may include hardware components such as a processing device(s) 414, which may include local or remote processing components. In some embodiments, the processing device(s) 414 may include any type of data processing capacity, such as a hardware logic circuit, for example an application specific integrated circuit (ASIC) and a programmable logic, or such as a computing device, for example, a microcomputer or microcontroller that include a programmable microprocessor. In some embodiments, the processing device(s) 414 may include data-processing capacity provided by the microprocessor. In some embodiments, the microprocessor may include memory, processing, interface resources, controllers, and counters. In some embodiments, the microprocessor may also include one or more programs stored in memory.

Similarly, the controller 410 may include data store 418, such as one or more local and/or remote data storage solutions such as, e.g., local hard-drive, solid-state drive, flash drive, database or other local data storage solutions or any combination thereof, and/or remote data storage solutions such as a server, mainframe, database or cloud services, distributed database or other suitable data storage solutions or any combination thereof. In some embodiments, the data store 418 may include, e.g., a suitable non-transient computer readable medium such as, e.g., random access memory (RAM), read only memory (ROM), one or more buffers and/or caches, among other memory devices or any combination thereof.

The data store 418 may store the values and/or parameters detailed above, including those associated with the dialysis process, pre-dialysis, patient, environment and/or operating characteristics of the dialysis system, among other values and/or parameters or any combination thereof. The values and/or parameters may be input manually via a user interface of the dialysis system, such as inputting patient characteristics, sorbent module parameters, fluids another data, including patient-specific values and/or parameters. Alternatively or in addition, one or more cartridge sensor(s) 110/210/310 of the recharge fluid circuit 100/200/300 may report values and/or parameters regarding detected effluent, dialysis and/or sorbent materials and properties, such as pH, sodium content, phosphate content, among other values and/or parameters as detailed above. Alternatively or additionally, the values and/or parameters may be loaded to the recharge control system 400 as a preset profile for a sorbent module. The values and/or parameters may be used as input to the recharge ML model(s) 412 for sorbent module state estimation and/or training of the recharge ML model(s) 412 parameters.

In some embodiments, the controller 410 may implement computer engines for the recharge ML model(s) 412 (e.g., inference and/or training). In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; ×86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

The controller 410 may determine current dialysis cartridge recharge characteristics associated with a dialysis cartridge recharge of a dialysis cartridge configured for dialysis therapy of the patient. For example, the recharge control system 400 can implement the controller 410 to determine a desired initial therapy sorbent material effluent pH. As described, the desired initial therapy sorbent material effluent pH can be based on one or more patient parameters and system parameters, including the patient's pre-treatment bicarbonate and urea levels, as well as the available of additional bicarbonate to be added during dialysis and the degassing capabilities of the system. The desired initial therapy sorbent material effluent pH can be determined by the recharge control system 400 based on the patient parameters and/or system parameters, or directly entered by a user through a user interface. The concentrations of acid, base, and sodium salt in the recharge solution can be determined using the cartridge sensor(s) 110/210/310. The described concentrations may depend on the pKa of the acid or buffer, the buffer capacity, and the temperature of the recharge solution, and can be automatically determined by the recharge control system 400. Where a single recharge solution source is used, the recharge control system 400 can automatically inform the user to add a specific amount of acid, base, or salt to the recharge solution. Where two or more recharge solution sources are used, the recharge control system 400 can determine the relative amounts of fluid needed from each recharge solution source.

Additionally or alternatively, the controller 410 can determine an amount of cations removed by the sorbent material module in a previous dialysis session. The amount of cations removed by the sorbent material module depends on the pre-dialysis patient potassium, calcium, magnesium, and urea levels of the patient, as well as patient weight, patient bicarbonate level, dialysate flow rate, blood flow rate, dialyzer size, dialysis time, ultrafiltration rate, and the potassium, calcium, magnesium, and bicarbonate dialysis prescription. The described patient parameters can automatically be received by the recharge control system 400 through a tracking device on the sorbent material module tracking usage. Alternatively, the described patient parameters can be input directly by the user based on the patient's medical records or other information. The described patient parameters can also be assumed by the system based on patient norms and settings entered into the system based on patient blood labs.

The controller 410 may receive current recharge fluid flow data 401 associated with a recharge process of sorbent material module. The current recharge fluid flow data 401 can include a fluid flow rate of the recharge solution(s) from the recharge solution source(s). Based on the fluid flow rate and an amount of time since the recharge process began, the controller 410 may draw a correlation to a degree of completion of the recharge process. As detailed above, analytically determining the completion status of the recharge process depends on numerous values and parameters related to the patient, environmental conditions, sensor sensitivity, types of fluids used by the sorbent, types of fluids used for the recharge solution, among other factors or any combination thereof. However, machine learning models may be implemented to infer a completion status of the recharge process non-deterministically to model complex relationships. Accordingly, the controller 410 can implement the recharge ML model(s) 412 to ingest the recharge fluid flow data 401, including flow rate, time/duration, among other fluid flow data, and output a prediction for the sorbent module state 416.

The recharge ML model(s) 412 may be configured to utilize one or more exemplary AI/machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neutral network technique may be one of, without limitation, feedforward neural network, radial basis function network, recurrent neural network, convolutional network (e.g., U-net) or other suitable network. Optionally, in combination of any embodiment described above or below, an exemplary implementation of Neural Network may be executed as follows:
a. define Neural Network architecture/model,
b. transfer the input data to the exemplary neural network model,
c. train the exemplary model incrementally,
d. determine the accuracy for a specific number of timesteps,
e. apply the exemplary trained model to process the newly-received input data,
f. optionally and in parallel, continue to train the exemplary trained model with a predetermined periodicity.

The exemplary trained neural network model may specify a neural network by at least a neural network topology, a series of activation functions, and connection weights. For example, the topology of a neural network may include a configuration of nodes of the neural network and connections between such nodes. The exemplary trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. The exemplary aggregation function may be a mathematical function that combines (e.g., sum, product, etc.) input signals to the node. An output of the exemplary aggregation function may be used as input to the exemplary activation function. The bias may be a constant value or function that may be used by the aggregation function and/or the activation function to make the node more or less likely to be activated.

The recharge ML model(s) 412 ingests a feature vector that encodes features representative of recharge fluid flow data 401. the recharge ML model(s) 412 processes the feature vector with parameters to produces a prediction of sorbent module state 416. The parameters of the recharge ML model(s) 412 may be implemented in a suitable machine learning model including a prediction machine learning model, such as, e.g., Linear Regression, Logistic Regression, Ridge Regression, Lasso Regression, Polynomial Regression, Bayesian Linear Regression (e.g., Naive Bayes regression), a convolutional neural network (CNN), a recurrent neural network (RNN), decision trees, random forest, support vector machine (SVM), K-Nearest Neighbors, or any other suitable algorithm for predicting output values based on input values. For computational efficiency while preserving accuracy of predictions, the recharge ML model(s) 412 may advantageously include a random forest model and/or linear regression.

The recharge ML model(s) 412 processes the features encoded in the feature vector by applying the parameters of the prediction machine learning model to produce a model output vector. the model output vector may be decoded to generate one or more numerical output values indicative of sorbent module state 416. The model output vector may include or may be decoded to reveal the output value(s) based on a modelled correlation between the feature vector and a target output. the numerical output may represent sorbent module state 416 including, e.g., percent completion, concentration of sorbent material, quantity of sorbent material, sorbent material effluent pH, among other characterizations of degree of recharge completed for the sorbent module or any combination thereof.

The parameters of the recharge ML model(s) 412 may be trained based on feedback from the cartridge sensor(s) 110/210/310 to calibrate the parameters of the recharge ML model(s) 412. For example, the recharge fluid flow data 401 may be paired with the sensed parameters (e.g., effluent pH, salt concentration in the effluent, phosphate concentration in the effluent, among others or any combination thereof) output by the cartridge sensor(s) 110/210/310. The sensed parameters indicate a state of the sorbent material module and thus can be used to test the accuracy of the sorbent module state 416 estimated by the recharge ML model(s) 412. Accordingly, an optimization function associated with the recharge ML model(s) 412 may compare the sensed parameters with the sorbent module state 416 to determine an error of the predicted output value the optimization function may employ a loss function, such as, e.g., Hinge Loss, Multi-class SVM Loss, Cross Entropy Loss, Negative Log Likelihood, or other suitable classification loss function to determine the error of the predicted output value based on the known output. Based on the error, or degree of deviation from the sensed parameters, the optimization function can refine the model parameters to better fit the actual state of the sorbent module.

Indeed, based on the error, the optimization function may update the parameters of the recharge ML model(s) 412 using a suitable training algorithm such as, e.g., backpropagation for a prediction machine learning model. Backpropagation may include any suitable minimization algorithm such as a gradient method of the loss function with respect to the weights of the prediction machine learning model. Examples of suitable gradient methods include, e.g., stochastic gradient descent, batch gradient descent, mini-batch gradient descent, or other suitable gradient descent technique. As a result, the optimization function may update the parameters of the recharge ML model(s) 412 based on the error of predicted labels in order to train the recharge ML model(s) 412 to model the correlation between recharge fluid flow data 401 and sorbent module state 416 in order to produce more accurate output values based on recharge fluid flow data 401. Optimization of the model parameters may occur periodically and/or continuously during the recharge process, thus enabling the recharge ML model(s) 412 to be refined during the recharge process to better estimate the state of the sorbent module.

Where the error or deviation of the sorbent module state 416 relative to the sensed parameters exceeds a predetermined threshold, a recharge fault may be generated. The recharge fault represents a fault either in the recharge ML model(s) 412 or the recharge fluid circuit 100/200/300 or both. For example, where the error or deviation exceeds the predetermined threshold, the fault may indicate that the recharge fluid circuit 100/200/300 is not operating as expected. This is because the continual or periodic calibration of the model parameters ensures that the recharge ML model(s) 412 models the effectiveness of the recharge fluid circuit 100/200/300 with increasing accuracy, so if the error exceeds the predetermined threshold, than the operation of the recharge fluid circuit 100/200/300 changed in an unexpected way, indicating a malfunction or fault.

The cartridge sensor(s) 110/210/310 may have a measurement error and sensitivity for which the cartridge sensor(s) 110/210/310 is designed. The recharge ML model(s) 412 can be configured to output sorbent module states 416 with any degree of precision/significant figures. Thus, the recharge ML model(s) 412 may generate sorbent module state 416 estimates with greater precision than the cartridge sensor(s) 110/210/310 are capable and/or at greater sampling frequencies than the sampling rate(s) of the cartridge sensor(s) 110/210/310. May generate more accurate measurements of the state of the sorbent module than the cartridge sensor(s) 110/210/310 is capable of, thus improving the accuracy of the control of the recharge process.

The processing device(s) 414 can receive, from at least one cartridge sensor(s) 110/210/310, a sorbent material module status representative of a quantity of materials and/or minerals remaining in the sorbent material module. Indeed, the processing device(s) 414 can determine the amount of acid, base, and sodium salt necessary to achieve the desired initial therapy sorbent material effluent pH based on the sorbent module state 416. Using the sorbent module state 416, the processing device(s) 414 can automatically determine the pump rates and/or valve switching necessary to recharge the sorbent material from one or more recharge solution sources and control the pumps and valves to generate the recharge solution to as to achieve the amount of acid, base and sodium salt for the desired initial therapy sorbent material effluent pH. The processing device(s) 414 controls the pumps and valves to recharge the sorbent material module.

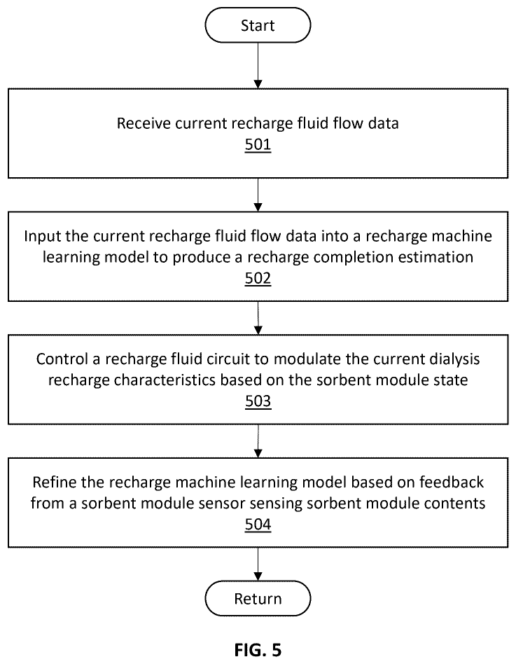

FIG. 5 is a flow diagram illustrating a recharge control method for recharging a sorbent module according to ML-based prediction of the sorbent module state in accordance with one or more embodiments of the present disclosure

At step 501, the controller 410 receives current recharge fluid flow data associated with a recharge process of sorbent material module. The current recharge fluid flow data 401 can include a fluid flow rate of the recharge solution(s) from the recharge solution source(s). Based on the fluid flow rate and an amount of time since the recharge process began, the controller 410 may draw a correlation to a degree of completion of the recharge process. As detailed above, analytically determining the completion status of the recharge process depends on numerous values and parameters related to the patient, environmental conditions, sensor sensitivity, types of fluids used by the sorbent, types of fluids used for the recharge solution, among other factors or any combination thereof. However, machine learning models may be implemented to infer a completion status of the recharge process non-deterministically to model complex relationships. Accordingly, the controller 410 can implement the recharge ML model(s) to ingest the recharge fluid flow data, including flow rate, time/duration, among other fluid flow data, and output a prediction for the recharge completion estimate.

At step 502, the controller 410 can input the current recharge characteristics into a recharge machine learning model to produce a sorbent module state indicative of a degree of recharge of the sorbent module that has been completed and/or a degree of recharging remaining in the recharge process. Indeed, the recharge ML model(s) may ingest and process the current recharge characteristics with model parameters to produces a prediction of the sorbent module state. The parameters of the recharge ML model(s) may be implemented in a suitable machine learning model including a prediction machine learning model, such as, e.g., Linear Regression, Logistic Regression, Ridge Regression, Lasso Regression, Polynomial Regression, Bayesian Linear Regression (e.g., Naive Bayes regression), a convolutional neural network (CNN), a recurrent neural network (RNN), decision trees, random forest, support vector machine (SVM), K-Nearest Neighbors, or any other suitable algorithm for predicting output values based on input values. For computational efficiency while preserving accuracy of predictions, the recharge ML model(s) 412 may advantageously include a random forest model and/or linear regression.

The recharge ML model(s) processes the current recharge characteristics by applying the parameters of the prediction machine learning model to produce a model output vector. the model output vector may be decoded to generate one or more numerical output values indicative of sorbent module state. The model output vector may include or may be decoded to reveal the output value(s) based on a modelled correlation between the feature vector and a target output. the numerical output may represent sorbent module state including, e.g., percent completion, concentration of sorbent material, quantity of sorbent material, sorbent material effluent pH, among other characterizations of degree of recharge completed for the sorbent module or any combination thereof.

At step 503, the controller 410 can control a recharge fluid circuit to modulate the current dialysis recharge characteristics based on the completion estimation. Using the sorbent module state produced by the recharge ML model(s), the controller 410 can automatically determine the pump rates and/or valve switching necessary to recharge the sorbent material from one or more recharge solution sources and control the pumps and valves to generate the recharge solution to as to achieve the amount of acid, base and sodium salt for the desired initial therapy sorbent material effluent pH.

At step 504, the controller 410 may refine the recharge machine learning model based on feedback from a dialysis cartridge sensor sensing cartridge contents. To do so, the parameters of the recharge ML model(s) may be trained based on feedback from the cartridge sensor(s) to calibrate the parameters of the recharge ML model(s). For example, the recharge fluid flow data 401 may be paired with the sensed parameters (e.g., effluent pH, salt concentration in the effluent, phosphate concentration in the effluent, among others or any combination thereof) output by the cartridge sensor(s). The sensed parameters indicate a state of the sorbent material module and thus can be used to test the accuracy of the sorbent module state estimated by the recharge ML model(s). Accordingly, an optimization function associated with the recharge ML model(s) may compare the sensed parameters with the sorbent module state to determine an error of the predicted output value. the optimization function may employ a loss function, such as, e.g., Hinge Loss, Multi-class SVM Loss, Cross Entropy Loss, Negative Log Likelihood, or other suitable classification loss function to determine the error of the predicted output value based on the known output. Based on the error, or degree of deviation from the sensed parameters, the optimization function can refine the model parameters to better fit the actual state of the sorbent module. As a result, the optimization function may update the parameters of the recharge ML model(s) based on the error of predicted labels in order to train the recharge ML model(s) to model the correlation between recharge fluid flow data and sorbent module state in order to produce more accurate output values based on recharge fluid flow data. Optimization of the model parameters may occur periodically and/or continuously during the recharge process, thus enabling the recharge ML model(s) to be refined during the recharge process to better estimate the state of the sorbent module.

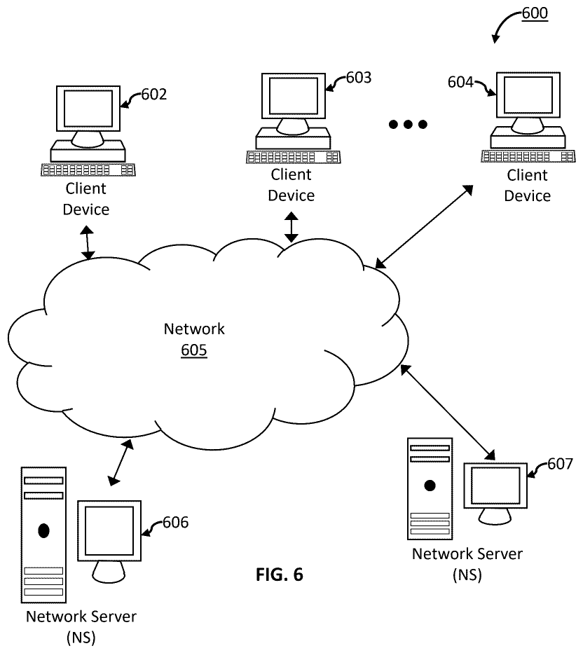

FIG. 6 depicts a block diagram of an exemplary computer-based system and platform 600 for ML-based sorbent module recharging in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 600 may be configured to manage a large number of members and concurrent transactions, as detailed herein. In some embodiments, the exemplary computer-based system and platform 600 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 6, client device 602, client device 603 through client device 604 (e.g., clients) of the exemplary computer-based system and platform 600 may include virtually any computing device capable of receiving and sending a message over a network (e.g., cloud network), such as network 605, to and from another computing device, such as servers 606 and 607, each other, and the like. In some embodiments, the client devices 602 through 604 may be or may be part of the recharge control system 400 detailed above, including, e.g., personal computers, multiprocessor systems, microprocessor-based or programmable consumer electronics, network PCs, and the like. In some embodiments, one or more client devices within client devices 602 through 604 may include computing devices that typically connect using a wireless communications medium such as cell phones, smart phones, pagers, walkie talkies, radio frequency (RF) devices, infrared (IR) devices, citizens band radio, integrated devices combining one or more of the preceding devices, or virtually any mobile computing device, and the like. In some embodiments, one or more client devices within client devices 602 through 604 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, OFDM, OFDMA, LTE, satellite, ZigBee, etc.). In some embodiments, one or more client devices within client devices 602 through 604 may include may run one or more applications, such as Internet browsers, mobile applications, voice calls, video games, videoconferencing, and email, among others. In some embodiments, one or more client devices within client devices 602 through 604 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a client device within client devices 602 through 604 may be specifically programmed by either Java, .Net, QT, C, C++, Python, PHP and/or other suitable programming language. In some embodiment of the device software, device control may be distributed between multiple standalone applications. In some embodiments, software components/applications can be updated and redeployed remotely as individual units or as a full software suite. In some embodiments, a client device may periodically report status or send alerts over text or email. In some embodiments, a client device may contain a data recorder which is remotely downloadable by the user using network protocols such as FTP, SSH, or other file transfer mechanisms. In some embodiments, a client device may provide several levels of user interface, for example, advance user, standard user. In some embodiments, one or more client devices within client devices 602 through 604 may be specifically programmed include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 605 may provide network access, data transport and/or other services to any computing device coupled to it. In some embodiments, the exemplary network 605 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 605 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 605 may include and implement, as an alternative or in conjunction with one or more of the above, a WIMAX architecture defined by the WiMAX forum. In some embodiments and, optionally, in combination of any embodiment described above or below, the exemplary network 605 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 605 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, OFDM, OFDMA, LTE, satellite and any combination thereof. In some embodiments, the exemplary network 605 may also include mass storage, such as network attached storage (NAS), a storage area network (SAN), a content delivery network (CDN) or other forms of computer or machine readable media.

In some embodiments, the exemplary server 606 or the exemplary server 607 may be a web server (or a series of servers) running a network operating system, examples of which may include but are not limited to Apache on Linux or Microsoft IIS (Internet Information Services). In some embodiments, the exemplary server 606 or the exemplary server 607 may be used for and/or provide cloud and/or network computing. Although not shown in FIG. 6, in some embodiments, the exemplary server 606 or the exemplary server 607 may have connections to external systems like email, SMS messaging, text messaging, ad content providers, etc. Any of the features of the exemplary server 606 may be also implemented in the exemplary server 607 and vice versa.

In some embodiments, one or more of the exemplary servers 606 and 607 may be specifically programmed to perform, in non-limiting example, as authentication servers, search servers, email servers, social networking services servers, Short Message Service (SMS) servers, Instant Messaging (IM) servers, Multimedia Messaging Service (MMS) servers, exchange servers, photo-sharing services servers, advertisement providing servers, financial/banking-related services servers, travel services servers, or any similarly suitable service-base servers for users of the client devices 601 through 604.

In some embodiments and, optionally, in combination of any embodiment described above or below, for example, one or more exemplary computing client devices 602 through 604, the exemplary server 606, and/or the exemplary server 607 may include a specifically programmed software module that may be configured to send, process, and receive information using a scripting language, a remote procedure call, an email, a tweet, Short Message Service (SMS), Multimedia Message Service (MMS), instant messaging (IM), an application programming interface, Simple Object Access Protocol (SOAP) methods, Common Object Request Broker Architecture (CORBA), HTTP (Hypertext Transfer Protocol), REST (Representational State Transfer), SOAP (Simple Object Transfer Protocol), MLLP (Minimum Lower Layer Protocol), or any combination thereof.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

As used herein, the term “runtime” corresponds to any behavior that is dynamically determined during an execution of a software application or at least a portion of software application.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, the NFC's peer-to-peer communication can be conducted when a plurality of NFC-enable devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors: ×86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) FreeBSD, NetBSD, OpenBSD; (2) Linux; (3) Microsoft Windows™; (4) Open VMS™; (5) OS X (MacOS™); (6) UNIX™; (7) Android; (8) iOS™; (9) Embedded Linux; (10) Tizen™; (11) WebOS™; (12) Adobe AIR™; (13) Binary Runtime Environment for Wireless (BREW™); (14) Cocoa™ (API); (15) Cocoa™ Touch; (16) Java™ Platforms, (17) JavaFX™; (18) QNX™; (19) Mono; (20) Google Blink; (21) Apple WebKit, (22) Mozilla Gecko™; (23) Mozilla XUL; (24) .NET Framework; (25) Silverlight™; (26) Open Web Platform; (27) Oracle Database; (28) Qt™; (29) SAP NetWeaver™; (30) Smartface™; (31) Vexi™; (32) Kubernetes™ and (33) Windows Runtime (WinRT™) or other suitable computer platforms or any combination thereof. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “proximity detection,” “locating,” “location data,” “location information,” and “location tracking” refer to any form of location tracking technology or locating method that can be used to provide a location of, for example, a particular computing device, system or platform of the present disclosure and any associated computing devices, based at least in part on one or more of the following techniques and devices, without limitation: accelerometer(s), gyroscope(s), Global Positioning Systems (GPS); GPS accessed using Bluetooth™; GPS accessed using any reasonable form of wireless and non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, Cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For case, at times the above variations are not listed or are only partially listed; this is in no way meant to be a limitation.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time: (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MDS, RIPEMD-160, RTRO, SHA-1, SHA-2, Tiger (TTH), WHIRLPOOL, RNGs).

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

The aforementioned examples are, of course, illustrative and not restrictive.

At least some aspects of the present disclosure will now be described with reference to the following numbered clauses.

1. A method, comprising:
[GT to complete upon finalization of claims]

Publications cited throughout this document are hereby incorporated by reference in their entirety. While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method comprising:
receiving, by at least one processor, current sorbent material module recharge characteristics associated with a sorbent material module recharge of a sorbent material module configured for dialysis therapy of a patient;
inputting, by the at least one processor, the current sorbent material module recharge characteristics into a recharge machine learning model to output a recharge completion estimation of the sorbent material module based at least in part on recharge machine learning model parameters;
wherein the recharge completion estimation represents a degree of sorbent material module recharge being complete; and
controlling, by the at least one processor, a sorbent material module recharge pump to vary the current sorbent material module recharge characteristics based at least in part on the recharge completion estimation.

2. The method of claim 1, further comprising:
receiving, by the at least one processor, from at least one sorbent material module sensor, at least one contents status representative of a quantity of contents remaining in the sorbent material module;
determining, by the at least one processor, a deviation between the recharge completion estimation and the at least one contents status; and
calibrating, by the at least one processor, the recharge machine learning model by updating the recharge machine learning model parameters based at least in part on the deviation between the recharge completion estimation and the at least one contents status.

3. The method of claim 2, wherein the contents comprises at least one of:
minerals, or
microbes.

4. The method of claim 1, wherein the recharge completion estimation comprises a precision greater than at least one contents status from at least one sorbent material module sensor configured to measure a quantity of contents remaining in the sorbent material module.

5. The method of claim 1, further comprising:
receiving, by the at least one processor, from at least one sorbent material module sensor, at least one contents status representative of a quantity of contents remaining in the sorbent material module;
determining, by the at least one processor, a deviation between the recharge completion estimation and the at least one contents status; and
determining, by the at least one processor, a recharge fault associated with the sorbent material module recharge based at least in part on the deviation exceeding a threshold deviation.

6. The method of claim 1, wherein the current sorbent material module recharge characteristics comprises at least one of:
a flow rate of a recharge fluid, or
a time associated with the sorbent material module recharge.

7. The method of claim 1, wherein the recharge machine learning model comprises at least one linear regression machine learning model.

8. The method of claim 1, further comprising:
determining, by the at least one processor, a quantity of recharge fluids needed to complete the sorbent material module recharge based at least in part on the recharge completion estimation; and
controlling, by the at least one processor, a sorbent material module recharge pump to vary the current sorbent material module recharge characteristics based at least in part on the quantity of recharge fluids needed.

9. A method comprising:
receiving, by at least one processor, current sorbent material module recharge characteristics associated with a sorbent material module recharge of a sorbent material module configured for dialysis therapy of a patient;
inputting, by the at least one processor, the current sorbent material module recharge characteristics into a recharge machine learning model to output a recharge completion estimation of the sorbent material module based at least in part on recharge machine learning model parameters;
wherein the recharge completion estimation represents a degree of sorbent material module recharge being complete;
receiving, by the at least one processor, from at least one sorbent material module sensor, at least one contents status representative of a quantity of contents remaining in the sorbent material module;
determining, by the at least one processor, a deviation between the recharge completion estimation and the at least one contents status; and
calibrating, by the at least one processor, the recharge machine learning model by updating the recharge machine learning model parameters based at least in part on the deviation between the recharge completion estimation and the at least one contents status.

10. The method of claim 9, further comprising:
controlling, by the at least one processor, a sorbent material module recharge pump to vary the current sorbent material module recharge characteristics based at least in part on the recharge completion estimation.

11. The method of claim 9, wherein the contents comprises at least one of:
minerals, or
microbes.

12. The method of claim 9, wherein the recharge completion estimation comprises a precision greater than at least one contents status from at least one sorbent material module sensor configured to measure a quantity of contents remaining in the sorbent material module.

13. The method of claim 9, further comprising:
receiving, by the at least one processor, from at least one sorbent material module sensor, at least one contents status representative of a quantity of contents remaining in the sorbent material module;
determining, by the at least one processor, a deviation between the recharge completion estimation and the at least one contents status; and
determining, by the at least one processor, a recharge fault associated with the sorbent material module recharge based at least in part on the deviation exceeding a threshold deviation.

14. The method of claim 9, wherein the current sorbent material module recharge characteristics comprises at least one of:
a flow rate of a recharge fluid, or
a time associated with the sorbent material module recharge.

15. The method of claim 9, wherein the recharge machine learning model comprises at least one linear regression machine learning model.

16. The method of claim 9, further comprising:
determining, by the at least one processor, a quantity of recharge fluids needed to complete the sorbent material module recharge based at least in part on the recharge completion estimation; and
controlling, by the at least one processor, a sorbent material module recharge pump to vary the current sorbent material module recharge characteristics based at least in part on the quantity of recharge fluids needed.

17. A system comprising:
a sorbent material module recharge pump configured to pump recharge fluids through a sorbent material module to recharge the sorbent material module; and
at least one processor operably connected to the sorbent material module recharge pump, wherein the at least one processor is configured to execute software instructions that cause the at least one processor to:
receive current sorbent material module recharge characteristics associated with a sorbent material module recharge of a sorbent material module configured for dialysis therapy of a patient;
input the current sorbent material module recharge characteristics into a recharge machine learning model to output a recharge completion estimation of the sorbent material module based at least in part on recharge machine learning model parameters;
wherein the recharge completion estimation represents a degree of sorbent material module recharge being complete; and
control a sorbent material module recharge pump to vary the current sorbent material module recharge characteristics based at least in part on the recharge completion estimation.

18. The system of claim 17, wherein the at least one processor is configured to execute the software instructions that further cause the at least one processor to:
receive, from at least one sorbent material module sensor, at least one contents status representative of a quantity of contents remaining in the sorbent material module;
determine a deviation between the recharge completion estimation and the at least one contents status; and
calibrate the recharge machine learning model by updating the recharge machine learning model parameters based at least in part on the deviation between the recharge completion estimation and the at least one contents status.

19. The system of claim 17, wherein the at least one processor is configured to execute the software instructions that further cause the at least one processor to:
receive, from at least one sorbent material module sensor, at least one contents status representative of a quantity of contents remaining in the sorbent material module;
determine a deviation between the recharge completion estimation and the at least one contents status; and
determine a recharge fault associated with the sorbent material module recharge based at least in part on the deviation exceeding a threshold deviation.

20. The system of claim 17, wherein the at least one processor is configured to execute the software instructions that further cause the at least one processor to:
determine a quantity of recharge fluids needed to complete the sorbent material module recharge based at least in part on the recharge completion estimation; and
control a sorbent material module recharge pump to vary the current sorbent material module recharge characteristics based at least in part on the quantity of recharge fluids needed.
